(19) United States
(12) Patent Application Publication
(10) Pub. No.: US 2010/0221197 A1
(43) Pub. Date: Sep. 2, 2010

(54) ANTIMICROBIAL AGENT AND EXTERNAL PREPARATION FOR SKIN CONTAINING THE SAME

(75) Inventors: Yasuo Tanaka, Osaka (JP); Shuji Kanatani, Osaka (JP)

Correspondence Address: VEDDER PRICE P.C., 222 N. LASALLE STREET, CHICAGO, IL, 60601, US

(73) Assignee: TAIYO CORPORATION, Osaka (JP)

(21) Appl. No.: 12/676,857

(22) PCT Filed: Aug. 22, 2008
(86) PCT No.: PCT/JP2008/064990

§ 371 Date: Mar. 5, 2010

(30) Foreign Application Priority Data
Nov. 27, 2007 (JP) 2007-305710

(51) Int. Cl. A61K 8/18; C07C 59/00; A61K 31/20; A61Q 11/00

(52) U.S. Cl. 424/49; 562/579; 514/560

(57) ABSTRACT

A novel antimicrobial agent which has a high degree of safety, an excellent compounding property and strong antimicrobial activity is provided. The antimicrobial agent is characterized by containing ricinoleic acid monoglyceride or diglycerine ricinoleic acid monoester as an active ingredient. The antimicrobial agent of the present invention is suitable as a compounding component of an antimicrobial object selected from a food product, a food packaging material, tableware, a perfume cosmetic, a cosmetic, an external preparation for skin, a skin washing agent, a disinfectant, a lotion for external use, an agent for hair, a wiping sterilization agent, a pharmaceutical, a quasi drug and a hygiene material for the oral cavity.

### BACKGROUND OF THE INVENTION

1. Field of the Invention

The present invention relates to an antimicrobial agent containing ricinoleic acid monoglyceride or diglycerine ricinoleic acid monoester as an active ingredient, and further relates to an external preparation for skin containing the antimicrobial agent and an antimicrobial method using the antimicrobial agent.

2. Description of the Related Art

Those having antimicrobial activity have been known among monoglycerides of middle chain fatty acids and monoglycerides of long chain unsaturated fatty acids (hereinafter, these are collectively referred to as “middle/long chain fatty acids monoglyceride”), and these have been used for an antimicrobial purpose against heat resistant spore-forming bacteria and yeast. It has been attempted to augment an antimicrobial effect by combining a middle/long chain fatty acid monoglyceride with a perfume such as an organic acid, hinokitiol, a benzoic acid, a salicylic acid, thymol, eugenol or bisabolol, diglycerine fatty acid ester, polyglycerine fatty acid ester, an amino acid quaternary ammonium salt, polylysine, ethanol, glycine or lysozyme (see Patent Documents 1 to 5).

Patent Document 1: Japanese Published Unexamined Patent Application No. 2005-179211

Patent Document 2: Japanese Published Unexamined Patent Application No. 2003-183105

Patent Document 3: Japanese Published Unexamined Patent Application No. 2003-12411

Patent Document 4: Japanese Published Unexamined Patent Application No. 2002-212021

Patent Document 5: Japanese Published Unexamined Patent Application No. 2001-17137

Patent Document 6: Japanese Published Unexamined Patent Application No. 2000-270821

Although, the aforementioned middle/long chain fatty acid monoglyceride has antimicrobial activity to some extent, its solubility in water and alcohol is low and a crystal is separated out because the middle/long chain fatty acid monoglyceride is fat-soluble. Thus, this is inappropriate for applying in a suitable additive amount to various foods and cosmetics.

Polyglycerine fatty acid ester, sucrose fatty acid ester and polyoxyethylene sorbitan fatty acid ester have relatively enhanced solubility in water, but their antimicrobial activity is relatively reduced.

In addition to the above antimicrobial agents, for example, phenol-based, benzoic acid-based, sorbic acid-based, organic halogen-based and benzimidazole-based bactericidal agents and metal ions such as silver, copper and zinc ions have been known as the antimicrobial agent, but many of these pose problems in safety.

On the other hand, naturally occurring antimicrobial agents include, for example, ethanol, polylysine, lysozyme, protamine, lactoferrin, glycine, chitosan, thymol, eugenol, an oil-based licorice root extract, an Angelica keiskei extract, a bamboo extract and a spice extract. However, these naturally occurring antimicrobial agents have a high degree of safety, but are less than satisfactory in terms of strength of antimicrobial activity.

### SUMMARY OF THE INVENTION

The present invention has been made in view of the above circumstances, and it is an object of the present invention to provide a novel antimicrobial agent which has a high degree of safety, an excellent compounding property and strong antimicrobial activity.

As a result of an extensive study for solving the above problems, the present inventors have found that ricinoleic acid monoglyceride and diglycerine ricinoleic acid monoester exhibit strong antimicrobial activity and excellent compounding property, and completed the present invention.

That is, the gist of the present invention is as follows.

[1] An antimicrobial agent containing ricinoleic acid monoglyceride or diglycerine ricinoleic acid monoester as an active ingredient.

[2] An external preparation for skin containing the antimicrobial agent according to [1] above.

[3] A method for enhancing an antimicrobial power of an antimicrobial object by compounding ricinoleic acid monoglyceride or diglycerine ricinoleic acid monoester into the antimicrobial object selected from a food product, a food packaging material, tableware, a perfume cosmetic, a cosmetic, an external preparation for skin, a skin washing agent, a disinfectant, a lotion for external use, an agent for hair, a wiping sterilization agent, a pharmaceutical, a quasi drug and a hygiene material for the oral cavity.

According to the present invention, the novel antimicrobial agent which has a high degree of safety, an excellent compounding property and strong antimicrobial activity is provided. In particular, regarding the antimicrobial activity, the antimicrobial agent of the present invention exhibits high antimicrobial activity against oral cavity bacteria such as Streptococcus mutans and Porphyromonas gingivalis, Staphylococcus aureus, Staphylococcus epidemidis, Corynebacterium xerosis, Bacillus subtilis, Bacillus cereus, Listeria monocytogenes and Propionibacterium acnes. Thus, the antimicrobial agent can prevent bacterial infection and food poisoning and be effectively applicable to various cases by compounding the antimicrobial agent into the antimicrobial object, e.g., a food product, a food packaging material, tableware, a perfume cosmetic, a cosmetic, an external preparation for skin, a skin washing agent, a disinfectant, a lotion for external use, an agent for hair, a wiping sterilization agent, a pharmaceutical, a quasi drug and a hygiene material for the oral cavity and the like, which are used on the skin and mucosa.

### BRIEF DESCRIPTION OF THE DRAWINGS

FIG. 1 is a graph showing results of a microbicidal test for Staphylococcus aureus; and

FIG. 2 is a graph showing results of a microbicidal test for Propionibacterium acnes.

### DETAILED DESCRIPTION OF THE PREFERRED EMBODIMENTS

The antimicrobial agent of the present invention is characterized by containing ricinoleic acid monoglyceride or diglycerine ricinoleic acid monoester as the active ingredient. Ricinoleic acid monoglyceride is a compound in which one molecule of ricinoleic acid is bound to one molecule of glycerine through an ester bond. Diglycerine ricinoleic acid monoester is a compound in which one molecule of ricinoleic acid is bound to one molecule of diglycerine through the ester bond.

When the antimicrobial agent contains ricinoleic acid monoglyceride or diglycerine ricinoleic acid monoester as the active ingredient, the antimicrobial agent may contain other fatty acid glycerine esters obtained by binding one or more molecules of the fatty acids having 8 to 24 carbon atoms to one molecule of a glycerine component such as glycerine, diglycerine and triglycerine through the ester bond in the range in which the antimicrobial activity is not impaired in addition to the active ingredient.

The fatty acid which composes the other fatty acid glycerine esters includes, for example, an octanoic acid, a nonanoic acid, a decanoic acid, an undecanoic acid, a dodecanoic acid, a tridecanoic acid, a tetradecanoic acid, a pentadecanoic acid, a hexadecanoic acid, a heptadecanoic acid, an octadecanoic acid, a myristoleic acid, a palmitoleic acid, an oleic acid, a linoleic acid, an α-linolenic acid, a γ-linolenic acid, an eicosapentaenoic acid, a docosapentaenoic acid, docosahexaenoic acid and a ricinoleic acid. When the above fatty acid glycerine esters are contained as the component in the antimicrobial agent, a content of ricinoleic acid monoglyceride or diglycerine ricinoleic acid monoester in the antimicrobial agent is preferably 1% by weight or more and more preferably 10% by weight or more.

Ricinoleic acid monoglyceride can be produced by a publicly known method, and, for example, the method for esterifying the ricinoleic acid and glycerine using a chemical catalyst or an enzyme (lipase) can be included. Diglycerine ricinoleic acid monoester can be produced in the same method as that for ricinoleic acidmonoglyceride, except that glycerine is replaced with diglycerine. In the present invention, the method of using lipase is preferable in terms of being capable of being produced under a mild condition in the above methods.

Lipase used as the catalyst is not particularly limited as long as it recognizes glycerides as a substrate. For example, monoglyceride lipase, mono/diglyceride lipase, triglyceride lipase, cutinase and esterase are included. Among them, lipase is preferable, and in particular, lipase which scarcely recognizes fatty acid triglyceride as the substrate and recognizes fatty acid monoglyceride and/or fatty acid diglyceride as the substrate is preferable. Such lipase includes monoglyceride lipase and mono/diglyceride lipase.

As such lipase, for example, lipase derived from a microorganism belonging to the genus Penicillium, genus Pseudomonas, genus Burkholderia, genus Alcaligenes, genus Staphylococcus, genus Bacillus, genus Candida, genus Geotrichum, genus Rhizopus, genus Rhizomucor, genus Mucor, genus Aspergillus or genus Pseudozyme is used. Lipase derived from the microorganism belonging to the genus Penicillium or the genus Bacillus is more preferable. These lipases are commercially available in general and easily obtainable.

A purified lipase (including crude purification and partial purification) may be used. Further, free lipase or lipase immobilized on a carrier such as an ion exchange resin, a porous resin, ceramics or calcium carbonate may be used.

An amount of lipase used for an esterification reaction may be appropriately determined depending on a reaction temperature, a reaction period of time, pressure (degree of reduced pressure) and the like, is not particularly limited thereto, but is preferably 1 unit (U) to 10,000 U per 1 g of a reaction mixture solution. One unit of an enzyme activity in the case of lipase refers to an enzyme amount which liberates 1 μmol of the fatty acid in one minute in hydrolysis of olive oil. In the case of monoglyceride lipase or mono/diglyceride lipase, one unit is the enzyme amount which liberates 1 μmol of oleic acid in one minute in the hydrolysis of oleic acid monoglyceride.

Ricinoleic acid used for the esterification reaction may be any form of a free type, a metal salt type and an ester type. In the present invention, the free type is preferable in terms of easy progress of the esterification reaction.

The amount of glycerine or diglycerine used for the esterification reaction is not particularly limited, and normally is preferably 1 to 10 times the molar amount and more preferably 1.5 to 5 times the molar amount based on 1 mol amount of free ricinoleic acid.

In the present invention, ricinoleic acid monoglyceride can be produced with good purity when the ricinoleic acid and glycerine are the reaction materials, and diglycerine ricinoleic acid monoester can be produced with good purity when ricinoleic acid and diglycerine are the reaction materials, by appropriately adjusting the reaction temperature, the reaction period of time, the pressure (degree of reduced pressure) and the like in the esterification reaction. The reaction temperature is preferably 30 to 60° C., the reaction period of time is preferably 30 to 60 hours, and the pressure is preferably 2 to 30 mmHg. In order to keep the activity of the lipase, it is preferable to add water in an amount of 0.3 to 3% by weight based on a total amount of ricinoleic acid and glycerine (or diglycerine).

The esterification reaction may be a stationary reaction, or may be performed with mixing the reaction solution by various stirring methods, a shaking method, an ultrasonic method, a blowing method of nitrogen and the like, a circulation mixing method using a pump etc., a mixing method using a valve or a piston etc., or the combination thereof.

As the method for isolating and purifying ricinoleic acid monoglyceride (or diglycerine ricinoleic acid monoester) from the reaction mixture solution, any method for isolation and purification can be adopted. The method for isolation and purification includes, for example, deacidification, washing with water, distillation, solvent extraction, ion exchange chromatography, thin layer chromatography, membrane separation and the like, and the combination thereof.

The antimicrobial agent of the present invention exhibits high antimicrobial activity against oral cavity bacteria such as Streptococcus mutans and Porphyromonas gingivalis, Staphylococcus aureus, Staphylococcus epidemidis, Corynebacterium xerosis, Bacillus subtilis, Bacillus cereus, Listeria monocytogenes and Propionibacterium acnes.

The antimicrobial agent of the present invention exhibits high antimicrobial activity against the above various bacterial species. Thus, when the antimicrobial agent of the present invention is compounded into an antimicrobial object such as, for example, a food product, a food packaging material, tableware, a perfume cosmetic, a cosmetic, an external preparation for skin, a skin washing agent, a disinfectant, a lotion for external use, an agent for hair, a wiping sterilization agent, a pharmaceutical, a quasi drug or a hygiene material for the oral cavity, the antimicrobial power of the antimicrobial object can be enhanced. The content of the antimicrobial agent in the antimicrobial object is normally 0.0001 to 50% by weight and preferably 0.001 to 10% by weight.

When the antimicrobial agent of the present invention is compounded into the above antimicrobial object, one or more other type of antimicrobial agents may be combined. Other type of antimicrobial agents which can be combined include, for example, cetylpyridinium chloride, dequalinium chloride, benzalkonium chloride, chlorohexidine, triclosan, isopropylmethylphenol, ofloxacin, iodine, sodium fluoride, benzoic acid-based, sorbic acid-based, organic halogen-based and benzimidazole-based microbicidal agents, metal ions such as silver and copper ions, lecithin, sucrose fatty acid ester, glycerine fatty acid ester, polyoxyethylene sorbitan fatty acid ester, ethanol, propylene glycol, polylysine, lysozyme, chitosan, thymol, eugenol, and plant extracts such as an oil-based licorice root extract, a mulberry bark extract, an Angelica keiskei extract, a spice extract and polyphenol etc.

A form of the antimicrobial agent of the present invention can be appropriately changed depending on the aforementioned antimicrobial object, and, for example, a granular form, a paste form, a solid form or a liquid form can be adopted.

When the antimicrobial agent of the present invention is compounded into the aforementioned antimicrobial object, a publicly known apparatus (paddle mixer, homomixer, homogenizer and the like) which can produce the aforementioned form can be used preferably. Since the antimicrobial agent of the present invention has an excellent compounding property, the antimicrobial agent is not separated out as a crystal from the produced various antimicrobial objects.

The antimicrobial agent of the present invention can also be compounded as an antimicrobial component of the external preparation for skin. This allows enhancement of the antimicrobial power of the external preparation for skin. The content of the antimicrobial agent in the external preparation for skin is normally 0.0001 to 50% by weight and preferably 0.001 to 10% by weight.

In addition to the antimicrobial agent of the present invention, the external preparation for skin according to the present invention may contain various optional ingredients, e.g., purified water, alcohols, an oil-based ingredient, a surfactant, a thickener, a preservative, a moisturizing agent, a powder, a perfume, a pigment, an emulsifier, a pH adjuster, ceramides, sterols, an antioxidant, a singlet oxygen scavenger, an ultraviolet light absorber, a whitening agent, an anti-inflammatory agent and other antimicrobial agents, which are used in the ordinary external preparations for skin.

Specifically, the oil-based ingredient includes liquid paraffin, petrolatum, solid paraffin, lanolin, lanolin fatty acid derivatives, dimethylpolysiloxane, higher alcohol higher fatty acid esters, fatty acids, long chain amide amines, and animal and plant fats and oils etc. The surfactant includes polyoxyethylene hardened castor oil, isostearyl glycerine ether, polyoxyethylene alkyl ether, glycerine fatty acid ester, polyethylene glycol, monostearic acid sorbitan, polyoxyethylene monostearic acid sorbitan, polyoxyethylene lauryl ether phosphate salts, N-stearoyl-N-methyl taurate salts, Lauryl phosphate, monomyristyl phosphate, monocetyl phosphate, polyoxyethylene lauryl ether sulfate salts, lauryl sulfate triethanolamine and polyoxyethylene lauryl ether sulfate triethanolamine, etc. The thickener includes water-soluble polymer compounds such as carboxyvinyl polymers, carboxymethylcellulose, polyvinyl alcohol, carrageenan and gelatin. The moisturizing agent includes propylene glycol, glycerine, sorbitol, xylitol and maltitol etc. The powder includes talc, sericite, mica, kaolin, silica, bentonite, zinc flower and isinglass etc.

The form of the external preparation for skin is not particularly limited, and a cream form, a gel form, a milky lotion form, a lotion form, an ointment form, a powder form, a poultice, a powder agent, a dropping agent, a patch agent and an aerosol agent, etc., can be adopted depending on its intended purpose.

When the antimicrobial agent of the present invention is compounded into the external preparation for skin, the publicly known apparatus (paddle mixer, homomixer, homogenizer and the like) which can produce the aforementioned form can be used preferably. Since the antimicrobial agent of the present invention has an excellent compounding property, the antimicrobial agent is not separated out as a crystal from the produced external preparation for skin.

### EXAMPLES

The present invention will be described in more detail below by Test Examples and the like, but the present invention is not limited thereto.

### 1. Synthesis Example of Ricinoleic Acid Monoglyceride

### 1-1. Immobilization of Lipase

A carrier (weak basic anion exchange resin, brand name: Duolite A-568K supplied by Sumika Chemtex Co., Ltd.) was stirred in 1/10 N NaOH for 30 minutes, filtrated, subsequently washed with ion exchange water and then pH-equilibrated by adding a 200 mM phosphate buffer (pH7.0). The phosphate buffer containing the pH-equilibrated carrier was subjected to ethanol substitution for 10 minutes. Then, in order to keep an enzyme activity, ricinoleic acid was adsorbed to the carrier using a solution of ricinoleic acid/ethanol=1/10 (weight ratio) for 20 minutes. Subsequently, the carrier to which the ricinoleic acid had been adsorbed was filtrated, and then washed by adding a 200 mM phosphate buffer (pH7.0) to the carrier. And, the carrier was filtrated and collected. 2 mL of a solution of lipase (derived from Penicillium camembertii, brand name: Lipase G supplied by Amano Enzyme Inc.) in an amount of 5000 U/mL based on 1 g of the carrier was brought into contact with the carrier for 2 hours to immobilize the lipase to the carrier. Finally, the carrier to which the lipase had been immobilized was filtrated and collected, and washed with ion exchange water to be used as an immobilized enzyme in subsequent reactions.

### 1-2. Synthesis Reaction

Into a vial of approximately 30 mL, 10 g of a mixed solution of the ricinoleic acid and glycerine (molar ratio=1/3), 0.1 g of water and 0.5 g of the immobilized enzyme prepared in “1-1. Immobilization of lipase” were added and reacted at 50° C. at 15 mmHg for 48 hours while stirring using a magnetic stirrer. After the reaction, a composition containing ricinoleic acid monoglyceride in an amount of 80% by weight in an oil layer was obtained. The resulting reaction product was repeatedly extracted on thin layer chromatographs to obtain purified ricinoleic acid monoglyceride in a content of 96%.

### 2. Synthesis Example of Diglycerine Ricinoleic Acid Monoester

Into a vial of approximately 30 mL, 10 g of a mixed solution of the ricinoleic acid and diglycerine (molar ratio=1/3), 0.1 g of water and 200 U of lipase (derived from Penicillium camembertii, brand name: Lipase G supplied by Amano Enzyme Inc.) were added and reacted at 40° C. at 5 mmHg for 48 hours while stirring using the magnetic stirrer. After the reaction, a composition containing diglycerine ricinoleic acid monoester in an amount of 71% by weight in an oil layer was obtained. The resulting reaction product was repeatedly extracted on thin layer chromatographs to obtain purified diglycerine ricinoleic acid monoester in a content of 96%.

### 3. Antimicrobial Test

3-1. Antimicrobial Effect on Corynebacterium xerosis or Staphylococcus aureus

0.5 mL of a previously sterilized medium (brand name: Brain Heart Infusion Liquid Medium supplied by Nihon Pharmaceutical Co., Ltd.) was added into a 96-well deep type microplate, and 0.5 mL of ricinoleic acid monoglyceride or diglycerine ricinoleic acid monoester of the present invention (synthesized in “1. Synthesis Example of ricinoleic acid monoglyceride” and “2. Synthesis Example of diglycerine ricinoleic acid monoester,” respectively) was added thereto and serially prepared so that the final concentration in the medium was 3 ppm, 6 ppm, 12 ppm, 25 ppm, 50 ppm, 100 ppm, 200 ppm and 400 ppm. 0.1 mL of each cultured bacterial medium of Corynebacterium xerosis (JCM 1971) or Staphylococcus aureus (JCM 2151) at approximately 1×10⁸CFU/mL was added to this sample solution, stirred, and then cultured at 37° C. under an aerobic condition for 24 hours. The antimicrobial effect was visually determined, and compared with test plots in which the microorganism had not been added. The test plot in which no turbidity due to growth of the microorganism was observed was determined as the test plot having the antimicrobial effect, and the lowest concentration required for inhibiting the growth of the microorganism (hereinafter referred to as a “growth inhibition minimum concentration”) was measured. As a Comparative Example, the growth inhibition minimum concentration of 4-isopropyl-3-methylphenol known as the antimicrobial agent having a broad antimicrobial spectrum was measured in the same way as in the above. Results are shown in Table 1.

3-2. Antimicrobial effect on Propionibacterium acnes

0.5 mL of the previously sterilized medium (brand name: Brain Heart Infusion Liquid Medium supplied by Nihon Pharmaceutical Co., Ltd.) was added to a 96-well deep type microplate, and 0.5 mL of ricinoleic acid monoglyceride or diglycerine ricinoleic acid monoester of the present invention (synthesized in “1. Synthesis Example of ricinoleic acid monoglyceride” and “2. Synthesis Example of diglycerine ricinoleic acid monoester,” respectively) was added thereto and serially prepared so that the final concentration in the medium was 3 ppm, 6 ppm, 12 ppm, 25 ppm, 50 ppm, 100 ppm, 200 ppm and 400 ppm. 0.1 mL of a cultured bacterial medium of Propionibacterium acnes (JCM 6425) at approximately 1×10⁸CFU/mL was added to this sample solution, and cultured at 37° C. under an anaerobic condition using a deoxidizer for 48 hours. The antimicrobial effect was visually determined, and the growth inhibition minimum concentration was measured. As a Comparative Example, the growth inhibition minimum concentration of 4-isopropyl-3-methylphenol known as the antimicrobial agent having a broad antimicrobial spectrum was measured in the same way as in the above. Results are shown in Table 1.

TABLE 1

| | minimum inhibitory concentration (ppm) | | |
|---|---|---|---|
| | | diglycerin | |
| | ricinoleic acid | ricinoleic acid | 4-isopropyl-3- |
| test strain | monoglyceride | monoester | methyl phenol |
| C. xerosis(JCM1971) | 25 | 50 | 200 |
| S. aureus(JCM2151) | 25 | 50 | 200 |
| P. acnes(JCM6425) | 50 | 100 | 200 |

From Table 1, it was shown that the growth inhibition minimum concentration of ricinoleic acid monoglyceride and diglycerine ricinoleic acid monoester was 1/2 to 1/8 of that of 4-isopropyl-3-methylphenol for all bacterial species of Corynebacterium xerosis, Staphylococcus aureus and Propionibacterium acnes. Therefore, it was found that ricinoleic acid monoglyceride and diglycerine ricinoleic acid monoester exhibited the stronger antimicrobial activity than 4-isopropyl-3-methylphenol. When the antimicrobial effect was compared between ricinoleic acid monoglyceride and diglycerine ricinoleic acid monoester, it was found that ricinoleic acid monoglyceride had the stronger antimicrobial effect.

### 3-3. Antimicrobial Effect on Other Bacterial Species

The growth inhibition minimum concentrations of ricinoleic acid monoglyceride and diglycerine ricinoleic acid monoester of the present invention (synthesized in “1. Synthesis Example of ricinoleic acid monoglyceride” and “2. Synthesis Example of diglycerine ricinoleic acid monoester,” respectively) for the 9 indicator bacterial species shown in Table 2 were measured in the same way as in “3. Antimicrobial test” described above. As Comparative Examples, using 4 kinds of fatty acid glycerides exhibiting the antimicrobial action and 4-isopropyl-3-methylphenyl, the growth inhibition minimum concentrations were measured in the same way as in the above. The results are shown in Table 2.

TABLE 2

| | minimum inhibitory concentration (ppm) | | | | | | |
|---|---|---|---|---|---|---|---|
| | | diglycerin | | diglycerin | | diglycerin | |
| | ricinoleic acid | ricinoleic acid | lauric acid | lauric acid | myristic acid | myristic acid | 4-isopropyl-3- |
| test strain | monoglyceride | monoester | monoglyceride | monoester | monoglyceride | monoester | methyl phenol |
| S. mutans(JCM5175) | 25 | 50 | 25 | 50 | 100 | 50 | 200 |
| P. gingivalis(JCM8525) | 6 | — | 13 | — | 50 | — | — |
| S. aureus(JCM2151) | 25 | 50 | 50 | 50 | 400 | 50 | 200 |
| S. epidermidis(JCM2414) | 25 | 50 | 25 | 100 | >400 | >400 | 200 |
| C. xerosis(JCM1971) | 25 | 25 | 100 | 200 | 400 | 100 | — |
| B. subtile(JCM2151) | 25 | 50 | 25 | 100 | >400 | 25 | — |
| B. cereus(JCM2152) | 12.5 | 50 | 25 | 100 | 400 | — | 200 |
| L. monocytogenes(JCM7671) | 25 | 50 | 25 | 200 | 200 | — | 200 |
| E. coli(JCM1649) | >400 | >400 | >400 | >400 | >400 | >400 | 400 |

From Table 2, ricinoleic acid monoglyceride and diglycerine ricinoleic acid monoester exhibited the growth inhibition minimum concentration equivalent to or lower than those of the 4 kinds of fatty acid glycerides and 4-isopropyl-3-methylphenyl for Streptococcus mutans and Porphyromonas gingivalis, Staphylococcus aureus, Staphylococcus epidemidis, Corynebacterium xerosis, Bacillus subtilis, Bacillus cereus and Listeria monocytogenes. In particular, ricinoleic acid monoglyceride exhibited the growth inhibition minimum concentration which was unexceptionally lower than all the Comparative Examples described above. Ricinoleic acid monoglyceride and diglycerine ricinoleic acid monoester did not exhibit antimicrobial activity against Escherichia coli among the 9 indicator bacterial species shown in Table 2.

### 4. Microbicidal Test

4-1. Microbicidal Test for Staphylococcus aureus

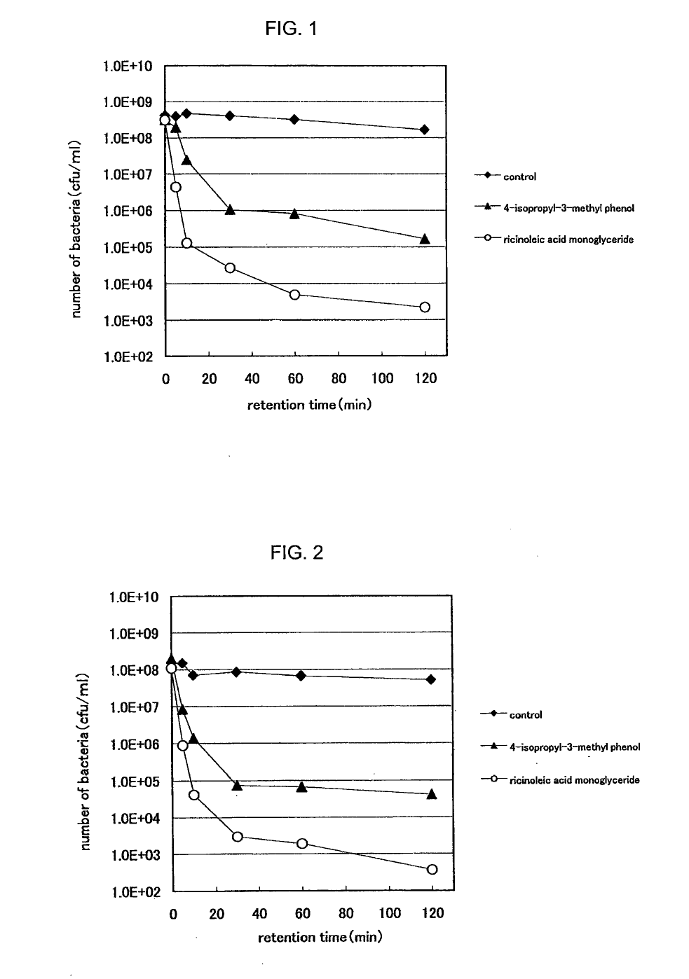

Ricinoleic acid monoglyceride synthesized in “1. Synthesis Example of ricinoleic acid monoglyceride” was added to a 0.2 M phosphate buffer (pH7.0) to prepare a 200 ppm sample. 0.1 mL of Staphylococcus aureus (JCM 2151) at approximately 1×10⁸CFU/mL was added to 5 mL of the sample. While being kept under the aerobic condition, the bacterial sample was taken at 0, 5, 10, 30, 60 and 120 minutes after the addition, and the number of survival bacteria in the sample was counted at each retention time. Specifically, using a Brain Heart Infusion agar medium, the sample was serially diluted, cultured using a flat plate smear method at 37° C. for 48 hours, and then the number of bacteria was counted. As a comparative control, 4-isopropyl-3-methylphenol was simultaneously evaluated in the same way as in the above. The results are shown in FIG. 1.

From FIG. 1, it was found that ricinoleic acid monoglyceride had the effect of reducing the number of bacteria to 1/1000 or less in 10 minutes and was useful as a fast-acting microbicidal agent. On the other hand, in 4-isopropyl-3-methylphenol which was the Comparative Example, the microbicidal action was weak for 10 minutes, and a retention time of 30 minutes was required for reducing the number of bacteria to 1/1000 or less.

4-2. Microbicidal Test for Propionibacterium acnes

Ricinoleic acid monoglyceride synthesized in “1. Synthesis Example of ricinoleic acid monoglyceride” was added to a 0.2 M phosphate buffer (pH7.0) to prepare a 200 ppm sample. 0.1 mL of Propionibacterium acnes (JCM 6425) at approximately 1×10⁸CFU/mL was added to 5 mL of the sample. While being kept under the anaerobic condition, the bacterial sample was taken at 0, 5, 10, 30, 60 and 120 minutes after the addition, and the number of survival bacteria in the sample was counted at each retention time. Specifically, using a GAM agar medium, the sample was serially diluted, cultured using the flat plate smear method at 37° C. under the anaerobic condition for 4 days, and then the number of bacteria was counted. As the comparative control, 4-isopropyl-3-methylphenol was simultaneously evaluated in the same way as in the above. The results are shown in FIG. 2.

From FIG. 2, ricinoleic acid monoglyceride reduced the number of bacteria to 1/100 or less in the retention time of a mere 5 minutes. On the other hand, in 4-isopropyl-3-methylphenol which was the Comparative Example, the microbicidal action was weak for 5 minutes, and a retention time of 10 minutes was required for reducing the number of bacteria to 1/100 or less.

### 5. Compounding Property

<Skin lotion>

| | |
|---|---|
| Hyaluronic acid (0.1% by weight aqueous solution) | 2.0% by weight |
| Glycerine | 5.0 |
| Ethanol | 5.0 |
| Ricinoleic acid monoglyceride | 0.5 |
| Purified water | Balance |

### (Process of Manufacture)

Hyaluronic acid, ethanol, glycerine and ricinoleic acid monoglyceride were mixed, and then purified water was added to obtain a skin lotion.

### (Compounding Property)

Ricinoleic acid monoglyceride was easily mixed with the other ingredients. No turbidity and precipitation were observed in the obtained skin lotion.

<Milky lotion>

| | |
|---|---|
| Squalane | 8.0% by weight |
| Jojoba oil | 2.0 |
| Bees wax | 0.5 |
| Sorbitan sesquioleate | 0.8 |
| Xanthan gum | 0.2 |
| 1,3-Butylene glycol | 6.0 |
| Ethanol | 4.0 |
| Ricinoleic acid monoglyceride | 1.0 |
| N-palm oil fatty acid acyl L- | 0.2 |
| arginine ethyl-DL-pyrrolidone | |
| carboxylate salt | |
| Purified water | Balance |

### (Process of Manufacture)

Squalane, jojoba oil, bees wax and sorbitan sesquioleate were mixed, heated to 70° C. and dissolved (this was a mixture A). On the other hand, xanthan gum, 1,3-butylene glycol, ethanol and ricinoleic acid monoglyceride were mixed at room temperature (this was a mixture B). Subsequently, the mixture A and the mixture B were mixed, heated to 60° C., and added little by little to the purified water in which the N-palm oil fatty acid acyl L-arginine ethyl-DL-pyrrolidone carboxylate salt had been added, and vigorously stirring to emulsify to obtain a milky lotion.

### (Blending Property)

Ricinoleic acid monoglyceride was immediately mixed with the other ingredients. No separation and precipitation were observed in the obtained milky lotion.

<Cream>

| | |
|---|---|
| Squalane | 10.0% by weight |
| Stearic acid | 8.0 |
| Bees wax | 2.0 |
| Stearyl alcohol | 5.0 |
| Ricinoleic acid monoglyceride | 2.0 |
| N-palm oil fatty acid acyl L- | 10.0 |
| arginine ethyl-DL-pyrrolidone | |
| carboxylate salt | |
| Purified water | Balance |

### (Process of Manufacture)

Squalane, stearic acid, bees wax, stearyl alcohol and ricinoleic acid monoglyceride were mixed, heated to 70° C. and dissolved. The N-palm oil fatty acid acyl L-arginine ethyl-DL-pyrrolidone carboxylate salt and the purified water were added little by little to these heated and dissolved oil-based ingredients, and these were stirred well to obtain a cream.

### (Compounding Property)

Ricinoleic acid monoglyceride was mixed very well with the other ingredients. No separation and precipitation were observed in the obtained cream.

Ricinoleic acid monoglyceride and diglycerine ricinoleic acid monoester according to the present invention have the high antimicrobial activity and the excellent compounding property, and thus are preferable as compounding components of the antimicrobial object selected from a food product, a food packaging material, tableware, a perfume cosmetic, a cosmetic, an external preparation for skin, a skin washing agent, a disinfectant, a lotion for external use, an agent for hair, a wiping sterilization agent, a pharmaceutical, a quasi drug and a hygiene material for the oral cavity.

What is claimed is:

1. An antimicrobial agent containing ricinoleic acid monoglyceride or diglycerine ricinoleic acid monoester as an active ingredient.

2. An external preparation for skin containing the antimicrobial agent according to claim 1.

3. A method for enhancing an antimicrobial power of an antimicrobial object by compounding ricinoleic acid monoglyceride or diglycerine ricinoleic acid monoester into the antimicrobial object selected from a food product, a food packaging material, tableware, a perfume cosmetic, a cosmetic, an external preparation for skin, a skin washing agent, a disinfectant, a lotion for external use, an agent for hair, a wiping sterilization agent, a pharmaceutical, a quasi drug and a hygiene material for the oral cavity.
